(19) United States
(12) Patent Application Publication
(10) Pub. No.: US 2007/0282312 A1
(43) Pub. Date: Dec. 6, 2007

(54) Ophthalmologic apparatus

(75) Inventor: Christian Rathjen, Bremen (DE)

Correspondence Address: OLIFF & BERRIDGE, PLC, P.O. BOX 19928, ALEXANDRIA, VA, 22320, US

(73) Assignee: SIE AG SURGICAL INSTRUMENT ENGINEERING, Port (CH)

(21) Appl. No.: 11/806,407

(22) Filed: May 31, 2007

Related U.S. Application Data
(60) Provisional application No. 60/809,351, filed on May 31, 2006.

(51) Int. Cl. A61B 18/18

(52) U.S. Cl. 606/4

(57) ABSTRACT

An ophthalmologic apparatus having a light projector for projecting femtosecond laser pulses for breaking down eye tissue, respectively focused onto a focal surface, comprises optical means for producing pulse processing areas (E) that are respectively formed on the focal surface by one of the femtosecond laser pulses, and have an area boundary (e) on the focal surface that deviates from a circular shape. By comparison with the conventional circular pulse processing areas, the noncircular pulse processing areas (E) are aligned with reference to a processing line (s) such that, given the same pulse spacing (p) relatively small overlap areas (B) arise from successive pulse processing areas (E). The energy irradiated into overlap areas (B) by successive femtosecond laser pulses is thereby reduced without bridges of tissue being left. In addition, given the same pulse spacing (p), it is possible to produce a wider incision track (4), and thus to reduce the overall treatment time for incision surfaces with a number of incisions tracks (4) juxtaposed in a row.

### TECHNICAL FIELD

The present invention relates to an ophthalmologic apparatus having a light projector for projecting femtosecond laser pulses for breaking down eye tissue. The present invention relates, in particular, to an ophthalmologic apparatus having a light projector for projecting femtosecond laser pulses for breaking down eye tissue, the femtosecond laser pulses respectively being projected onto a focal surface.

### PRIOR ART

The particular feature of femtolaser systems, which exhibit pulse widths of typically 10 fs to 1000 fs (1 fs=10⁻¹⁵ s), is that it is also possible to treat transparent materials at the focus by means of nonlinear absorption and subsequent interaction (for example photodisruption). It is a characteristic of femtolaser systems that it is possible to guide the focus freely in the material, something which enables the production of any desired incision patterns. The surgical incision of the cornea that has been introduced in practice may, in particular, be adduced as an example; for which purpose a number of laser pulses are juxtaposed in a row one behind the other or next to one another by means of a scanning device (scanner). In order to avoid bridges of tissue being left during incision operations, the individual laser pulses are positioned by the scanning device such that the treatment areas of successive laser pulses overlap. However, the energy irradiated into the overlap area by a subsequent laser pulse does not contribute to the breakdown of tissue required for the incision. The energy deposited in the overlap area by the subsequent laser pulse is lost without being used and/or imposes a burden in an undesired way (heating) on the tissue adjoining the incision.

An apparatus for material treatment by means of nonlinear laser radiation is described in patent application WO 2005/092259. According to WO 2005/092259, a polarization modulator and/or an intensity modulator in the beam path is used to reduce the size of focus in order to avoid collateral damage in neighboring material areas. However, there still remains the disadvantage adduced above in the case of overlapping treatment areas of successive laser pulses.

### SUMMARY OF THE INVENTION

It is an object of the present invention to propose an ophthalmologic apparatus having a light projector for projecting femtosecond laser pulses for breaking down eye tissue that does not exhibit the disadvantages of the prior art. It is, in particular, an object of the present invention to propose an ophthalmologic apparatus having a light projector for projecting femtosecond laser pulses for breaking down eye tissue that enables the reduction of the undesired irradiation of energy into overlap zones of successive laser pulses without thereby giving rise to bridges of tissue that are left.

In accordance with the present invention, these aims are achieved, in particular, by means of the elements of the independent claims. Further advantageous embodiments emerge, furthermore, from the dependent claims and description.

The above-named aims are achieved by the present invention by virtue of the fact that, in particular, an ophthalmologic apparatus that comprises a light projector for projecting femtosecond laser pulses for breaking down eye tissue, respectively focused onto a focal surface (for example a focal plane or a convex focal surface), is provided with optical means for producing pulse processing areas that are respectively formed on the focal surface by one of the femtosecond laser pulses and respectively have an area boundary on the focal surface that deviates from a circular shape. The ophthalmologic apparatus is, for example, configured to produce incisions in the eye tissue. The area boundary is, for example, defined by a limit value for the drop in intensity of one of the laser pulses on the focal surface, that is to say the area boundary of a pulse processing area is fixed where the intensity of the irradiated light on the focal surface drops by or to at least the defined limit value, for example by 60% or, respectively, to 40%. The pulse processing areas produced have, for example, an elliptically or ovally shaped area boundary, and preferably exhibit a wider extent in a longitudinal direction than in a transverse direction, with a narrower extent, normal to the longitudinal direction. By comparison with the conventional circular pulse processing areas, the pulse processing areas produced are preferably extended in the longitudinal direction and compressed in the transverse direction. By comparison with conventional circular pulse processing areas, the pulse processing areas produced according to the invention can be aligned with reference to a processing line such that, given the same pulse spacing, smaller overlap areas are produced by successive pulse processing areas. This reduces the undesired irradiation of energy into overlap zones of successive laser pulses, without bridges of tissue thereby being left. In addition, the pulse processing areas produced according to the invention can be aligned with reference to the treatment direction such that, given the same pulse spacing, a wider incision track is produced. In the case of the production of incision surfaces by a number of incision tracks juxtaposed in a row, it is possible thereby to reduce the overall treatment time (incision time). In addition, there is also an overall reduction in the energy irradiated into the eye tissue during the eye treatment.

It is preferred for the pulse processing areas to be positioned along the processing line by positioning means, and for the optical means to be configured to align the pulse processing areas with reference to the processing line. In the case of straight processing lines, the optical means are permanently set to align the pulse processing areas with reference to the processing line. In the case of curved processing lines, for example of circular or spiral shape, the setting of the optical means for aligning the pulse processing areas with reference to the processing line is dynamically varied. The optical means are, for example, configured to align the pulse processing areas with reference to the processing line (doing so in a permanent or variable fashion) such that a longitudinal axis running in the wider extent of the pulse processing area respectively lies perpendicular to the processing line. As set forth above, a transverse position of the elongated extent of the pulse processing areas relative to the processing line gives rise to smaller overlap areas of the pulse processing areas of successive laser pulses, the result being to reduce undesired irradiation of energy into overlap zones without bridges of tissue thereby being left. Moreover, a wider incision track can be produced, the result being to reduce the incision time for an incision surface. By way of example, the optical means are configured as an alternative or in addition to align the pulse processing areas with reference to the processing line (in a permanent or variable fashion) such that a transverse axis running in the narrower extent of the pulse processing area respectively lies perpendicular to the processing line. Owing to the longitudinal alignment of the elongated extent of the pulse processing areas relative to the processing line, bridges of tissue that are left between pulse processing areas of successive laser pulses can be avoided when, for example, the deflection speed of a scanner of the positioning means is too high for the pulse rate of the laser that generates the femtosecond laser pulses. In addition, the longitudinal alignment of the elongated extent of the pulse processing areas, but also the transverse direction of the compressed extent of the pulse processing areas can be used in alternative applications in which a strong overlapping between successive pulse processing areas is desired, for example processing methods that place more than one laser pulse onto the same site in order to ablate tissue. Such methods operate with very high pulse rates (MHz) and relatively low pulse energies in order to use the accumulated effect of temporally successive laser pulses. By producing pulse processing areas with an area boundary deviating from a circular shape, and by means of the targeted alignment of the pulse processing areas with reference to the processing line, it is possible for lasers and positioning means, in particular scanner modules, to be tuned to one another and/or to be used in an optimized fashion more effectively, and this is advantageous, for example, when the frequency of a femtolaser source cannot be set to its fundamental frequency, or can be so set only in integral ratios (by pulse selection).

In a design variant, the ophthalmologic apparatus comprises a control module that is configured to control the optical means in such a way that the optical means align the pulse processing areas with reference to the processing line in accordance with a control value, and/or produce the shape of the area boundary in accordance with the control value. Such a control enables the shape and alignment of the pulse processing areas, and/or the size of the overlap areas or degree of overlap to be set flexibly.

The positioning means and the optical means are preferably configured to position or to produce the pulse processing areas such that successive pulse processing areas at least partially overlap so that no undesired bridges of tissue are left between successive pulse processing areas.

The optical means for producing the pulse processing areas preferably comprise an optical modulator, in particular a polarization modulator, a phase modulator and/or an intensity modulator, that is to say an optical modulator for modulating the radiation intensity, the phase delay times and/or the polarity over the beam cross section of the femtosecond laser pulses. The optical means for producing the pulse processing areas comprise, for example, one or more rotatable optical elements, for example anamorphotic optical modules, deformable mirrors, photonic crystals, photonic bandgap fibers, diffractive optical modules, lens arrays, polarization filters, spatial polarization plates, λ-half-plates, stops, and/or one or more spatial light modulators, for example LCD arrays or DLP (Digital Light Processing) projectors (mirror arrays).

### BRIEF DESCRIPTION OF THE DRAWINGS

A design of the present invention is explained below with the aid of an example. The example of the design is illustrated by the following enclosed Figures:

FIG. 1 shows a block diagram that represents schematically an ophthalmologic apparatus used in treating an eye by means of a focused pulsed laser beam.

FIGS. 2a, 2b, 2c and 2d respectively show a block diagram that illustrates schematically a design variant of the ophthalmologic apparatus used in treating an eye by means of a focused pulsed laser beam.

FIG. 3a shows a pulse processing area, formed by a femtosecond laser pulse on the focal surface and having a circular area boundary in accordance with the prior art.

FIG. 3b shows a number of pulse processing areas succeeding one another along a processing line and respectively having a circular area boundary in accordance with the prior art.

FIG. 4a shows a pulse processing area formed by a femtosecond laser pulse on the focal surface and having an area boundary deviating from the circular shape.

FIG. 4b shows a number of pulse processing areas succeeding one another along a straight processing line and having an area boundary deviating from the circular shape, and a longitudinal alignment transverse to the processing line.

FIG. 4c shows an alignment of a pulse processing area having an area boundary deviating from the circular shape along a curved processing line.

FIG. 5a shows a number of pulse processing areas succeeding one another along a straight processing line and having an area boundary deviating from the circular shape, and a longitudinal alignment along the processing line.

FIG. 5b shows a further alignment of a pulse processing area having an area boundary deviating from the circular shape along the curved processing line.

FIG. 6 shows a number of incision tracks juxtaposed in a row that are respectively formed by pulse processing areas succeeding one another along the straight processing line.

### WAYS OF IMPLEMENTING THE INVENTION

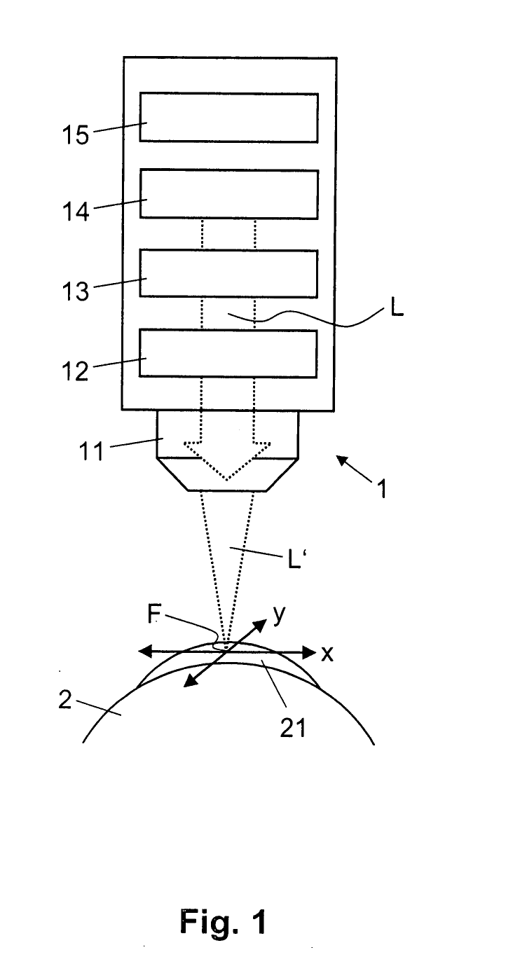

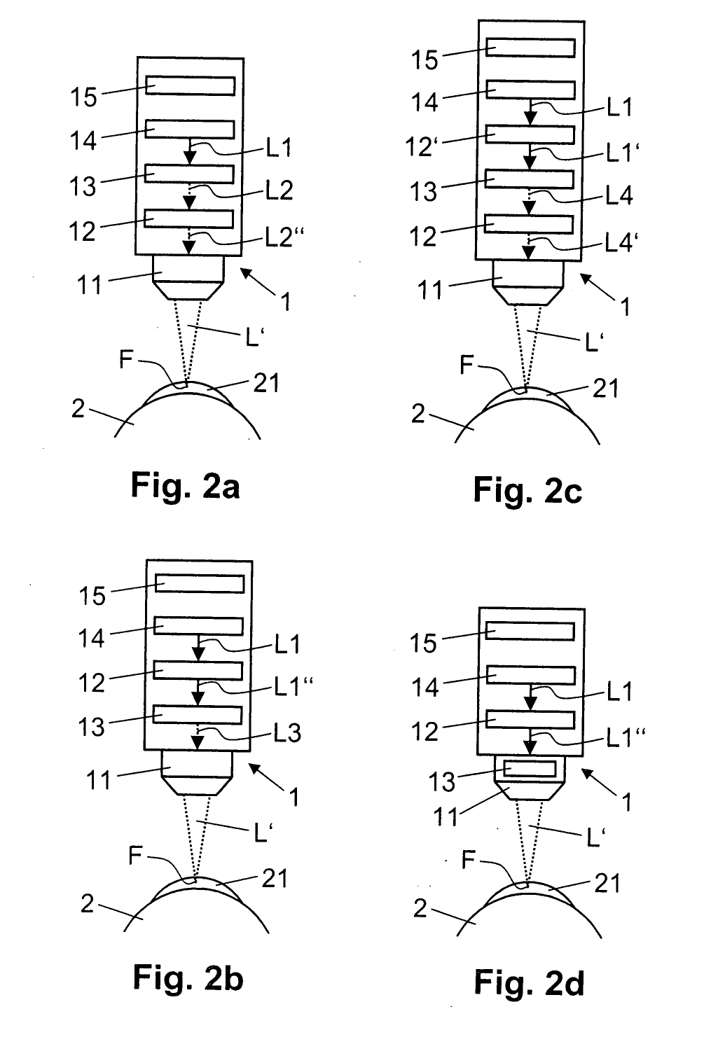

In FIGS. 1, 2a, 2b, 2c and 2d, the reference symbol 1 denotes an ophthalmologic apparatus having a laser source 14 and a light projector 11, optically connected to the laser source 14, for the generation and focused projection of a pulsed laser beam L1 for the punctiform breakdown of tissue at a focus F (focal point) in the interior of the eye tissue 21, for example in the cornea. The laser source 14 comprises a femtolaser for generating femtosecond laser pulses that have pulse widths of typically 10 fs to 1000 fs (1 fs=10⁻¹⁵ s). The laser source 14 is arranged in a separate housing or in a common housing with the light projector 11. As illustrated schematically in FIG. 1, the ophthalmologic apparatus 1 comprises positioning means 12 in order to move the focus F of the pulsed laser beam L′ in at least two dimensions x, y of a processing area (coherent or not coherent) in or on the tissue of the eye 3. In a design variant, the positioning means 12 are, however, configured also to move the focus F in a third direction, normal to the two dimensions x, y. In a design variant, the positioning and movement of the focus F is effected solely by an optical scanner module that appropriately deflects the pulsed light beam generated by the laser source 14. In a design variant, in addition to the optical scanner module, the positioning means 12 comprise one or more movement drivers for moving the light projector 11. The positioning means 12 are designed, for example, as in EP 1 486 185 (also incorporated by reference), and in a design variant superpose by means of optical microscans an additional fine movement on the translational movement of the focus caused by the movement of the light projector 11, doing so in accordance with EP 1 627 617. In European patent application No. 05 405 376 (not yet published) there is a description of a scanner module for deflecting a pulsed light beam for the additional fine movement, as well as of an optical transmission system for transmitting the deflected femtosecond laser pulses from the scanner module to the light projector 11 and for superposing the deflected femtosecond laser pulses on the movement of the light projector 11.

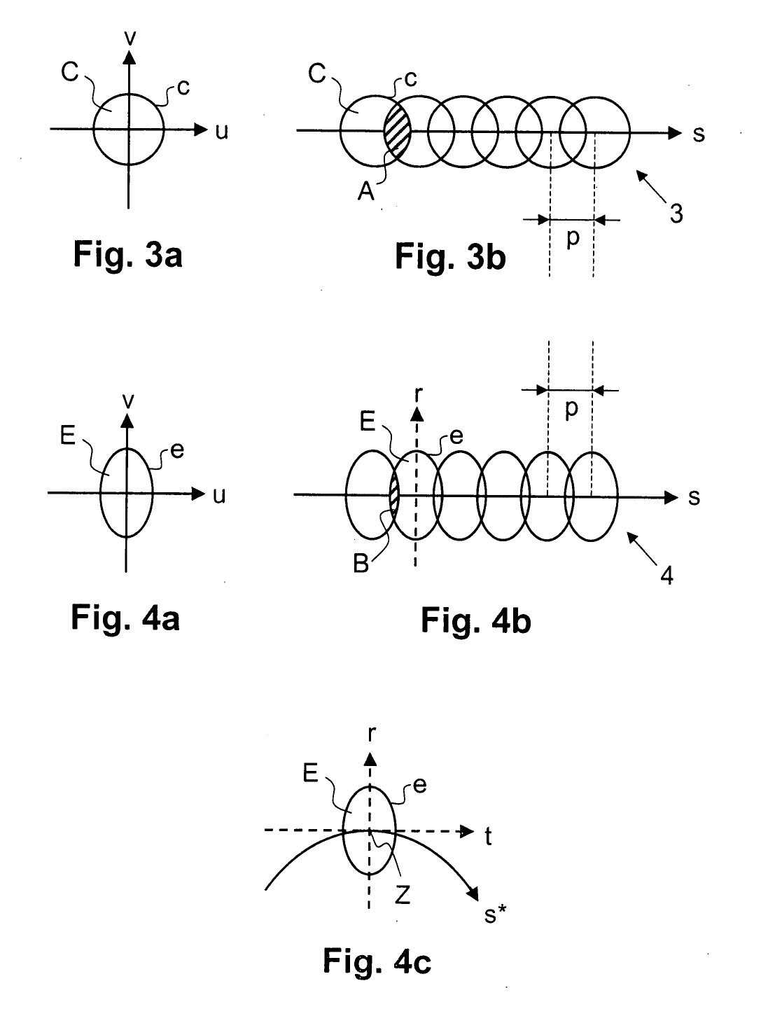

Owing to the focused projection of the pulsed laser beam L′, the femtosecond laser pulses respectively form pulse processing areas on the focal surface (that is to say focal plane or convex focal surface). The area boundary of a pulse processing area is defined, for example, by a limit value G for the drop in intensity 1/e² of the femtosecond laser pulse focused on the focal surface. The area boundary is, for example, defined where the intensity of the irradiated light on the focal surface drops by or to at least the defined limit value G, for example by 60% or to 40%. In FIGS. 3a and 4a, the reference symbols u and v denote the coordinate axes of the focal surface coinciding with the plane of a drawing. As is illustrated in FIG. 3a, a pulse processing area C, produced by a femtosecond laser pulse of a known system, on the focal surface exhibits a circular area boundary c. FIG. 3b shows an incision track 3 that has a number of pulse processing areas C succeeding one another along the straight processing line s. As is to be seen from FIG. 3b, the pulse processing areas (C, which are formed by successive femtosecond laser pulses, have overlap areas A.

As is illustrated in FIG. 1, the ophthalmologic apparatus 1 additionally comprises optical means 13 that are arranged in the schematically illustrated beam path L between the laser source 14 and the exit of the light projector 11. The optical means 13 are configured to influence the radiation intensity, the phase delay time and/or polarity over the beam cross section of the femtosecond laser pulses such that the femtosecond laser pulses form on the focal surface pulse processing areas E with an area boundary e deviating from the circular shape, as is illustrated in FIG. 4a, for example. By comparison with the conventional circular pulse processing areas C, the pulse processing areas E produced by the optical means 13 preferably extend in a longitudinal direction and are compressed in a transverse direction, and have an elliptically or ovally shaped area boundary e, for example. In a design variant, the laser source 14 is configured to generate femtosecond laser pulses with an elliptically or ovally shaped area boundary e deviating from the circular shape (occasioned by a typical Gaussian profile). In this last-named design variant, the optical means 13 are just configured to image or project the femtosecond laser pulses generated by the laser source 14 onto the focal surface in a focused fashion via the light projector 11, in order to produce pulse processing areas E with an elliptically or ovally shaped area boundary (e) on the focal surface.

In order to produce pulse processing areas E deviating from the circular shape, the optical means 13 comprise a polarization modulator, a phase modulator and/or an intensity modulator for modulating the radiation intensity, the phase delay times and/or the polarity over the beam cross section of the femtosecond laser pulses. The optical means 13 comprise, for example, an anamorphotic optical module (for example, movable spherical and/or cylindrical lenses), one or more deformable mirrors, photonic crystals, photonic “bandgap” fibers, diffractive optical modules, lens arrays, polarization filters, spatial polarization plates, λ-half-plates, stops, and/or one or more spatial light modulators, for example LCD arrays or DLP projectors. In the design variant according to FIG. 2a, the optical means 13 are inserted as optical module into the beam path between the laser source 14 and the positioning means 12. The femtosecond laser pulses L1 generated by the laser source 14 are fed to the optical means 13, which modulate the radiation intensity, the phase delay times and/or the polarity over the beam cross section of the femtosecond laser pulses L1 in order to produce noncircular pulse processing areas E. The femtosecond laser pulses L2 generated by the optical means 13 and having a modulated radiation intensity, phase delay time and/or polarity are fed to the positioning means 12. The positioning means 12 deflect the femtosecond laser pulses L2 into at least one scanning direction, and/or move the light projector 11 into one or two further scanning directions. The femtosecond laser pulses L2″ deflected by the positioning means 12 are fed to the light projector 11 for the purpose of focused projection.

In the design variant according to FIG. 2b, the optical means 13 are inserted as optical module into the beam path between the positioning means 12 and the light projector 11. The femtosecond laser pulses L1 generated by the laser source 14 are fed to the positioning means 12, which deflect the femtosecond laser pulses L1 into at least one scanning direction, and/or move the light projector 11 into one or two further scanning directions. The femtosecond laser pulses L1″ deflected by the positioning means 12 are fed to the optical means 13, which modulate the radiation intensity, the phase delay time and/or the polarity over the beam cross section of the femtosecond laser pulses L1″ in order to produce noncircular pulse processing areas E. The femtosecond laser pulses L3 generated by the optical means 13 and having a modulated radiation intensity, phase delay time and/or polarity are fed to the light projector 11 for the purpose of focused projection.

In the design variant according to FIG. 2c, the optical means 13 are inserted as optical module into the beam path between two modules of the positioning means 12, 12′. The femtosecond laser pulses L1 generated by the laser source 14 are fed to the first module of the positioning means 12′, which deflect the femtosecond laser pulses L1 into at least: one scanning direction. The femtosecond laser pulses L1′ deflected by the positioning means 12 are fed to the optical means 13, which modulate the radiation intensity, phase delay time and/or the polarity over the beam cross section of the femtosecond laser pulses L1′ in order to produce noncircular pulse processing areas E. The femtosecond laser pulses L4 generated by the optical means 13 and having modulated radiation intensity, phase delay time and/or polarity are fed to the second module of the positioning means 12, which deflect the femtosecond laser pulses 14 into at least one further scanning direction, and/or move the light projector 11 into one or two further scanning directions. The femtosecond laser pulses L4′ deflected by the second module of the positioning means 12 are fed to the light projector 11 for the purpose of focused projection.

In the design variant according to FIG. 2d, the optical means 13 are integrated in the light projector 11. The femtosecond laser pulses L1 generated by the laser source 14 are fed to the positioning means 12, which deflect the femtosecond laser pulses L1 in at least one scanning direction, and/or move the light projector 11 into one or two further scanning directions. The femtosecond laser pulses L1″ deflected by the positioning means 12 are fed to the optical means 13 in the light projector 11, which modulate the radiation intensity, the phase delay time and/or the polarity over the beam cross section of the femtosecond laser pulses L1″ in order to produce noncircular pulse processing areas E. The femtosecond laser pulses generated by the optical means 13 and having a modulated radiation intensity, phase delay time and/or polarity are projected by the light projector 11 in a focused fashion.

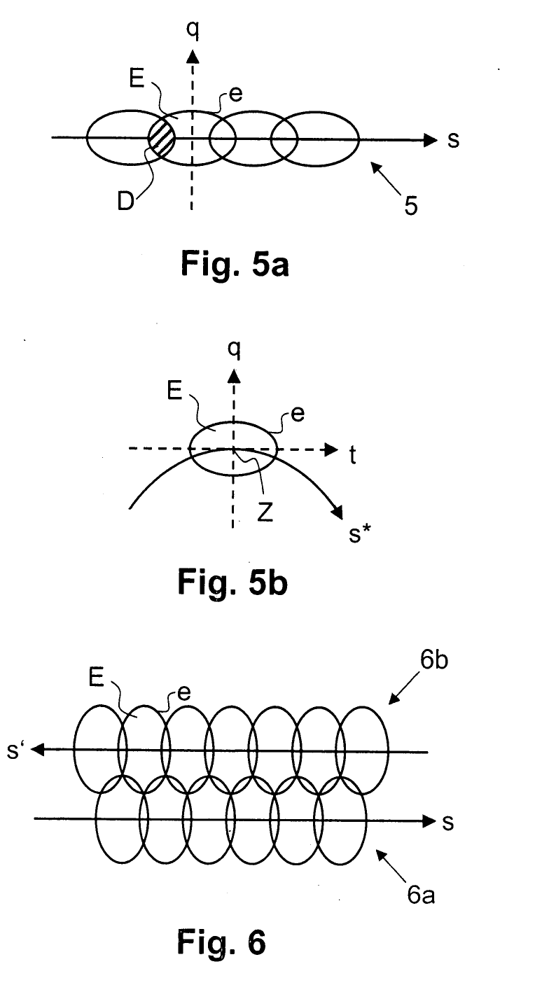

In FIGS. 1, 2a, 2b, 2c and 2d, the reference symbol 15 denotes a control module that is designed as a programmed logic module by means of software and/or hardware. The control module 15 is configured to control the positioning means 12 and the optical means 13. The control module 15 is configured, in particular, to control the optical means 13 in order to align the pulse processing areas E with reference to the processing line s. In a design variant, the control module 15 is, however, configured to control the optical means 13 in order to produce pulse processing areas E having a different shape, deviating from the circular shape. The control of the optical means 13 with regard to the alignment and/or shape of the pulse processing areas E is preferably based on one or more user-specific control values that define the shape of the pulse processing areas E, the size of the overlap areas B, D (see FIG. 5a), or a degree of overlap, and/or the processing pattern.

FIG. 4b shows an incision track 4 that has a number of pulse processing areas E which succeed one another along the straight processing line s, have an area boundary E in accordance with FIG. 4a deviating from the circular shape, and are aligned such that a longitudinal axis r running in the wider extent of the pulse processing area E respectively lies perpendicular to the processing line s. As is to be seen from FIG. 4b, the pulse processing areas E that are formed by successive femtosecond laser pulses of the same pulse spacing p as in FIG. 3b have smaller overlap areas B than the circular pulse processing areas C in FIG. 3b.

FIG. 4c shows an alignment of a pulse processing area E that has an area boundary e in accordance with FIG. 4a along a curved processing line s*. As may be seen from FIG. 4c, the pulse processing area E is aligned such that the longitudinal axis r running in the wider extent of the pulse processing area E lies perpendicular to the tangent t to the curved processing line s*, the center point Z of the pulse processing area E corresponding to the point of contact of the tangent t.

FIG. 5a shows an incision track 5 that has a number of pulse processing areas E, which follow one another along the straight processing line s, have an area boundary e in accordance with FIG. 4a that deviates from the circular shape, and are aligned such that a transverse axis q running in the narrower extent of the pulse processing area E respectively lies perpendicular to the processing line s.

FIG. 5b shows an alignment of a pulse processing area E, which has an area boundary e in accordance with FIG. 4a, along a curved processing line s*. As may be seen from FIG. 5b, the pulse processing area E is aligned such that the transverse axis q running in the narrower extent of the pulse processing area E lies perpendicular to the tangent t to the curved processing line s*, the center point Z of the pulse processing area E corresponding to the point of contact of the tangent t.

FIG. 6 shows a number of incision tracks 6a, 6b, juxtaposed in a row, in accordance with FIG. 4b. As may be seen from FIG. 6, the pulse processing areas E of the first incision track 6a, along the processing line s, and the pulse processing areas E of the second incision track 6a, along the processing line s′, are phase shifted such that no bridges of tissue are left between the incision tracks 6a, 6b, and the smallest possible overlap areas are formed by the pulse processing areas E of neighboring incision tracks 6a, 6b.

Finally, it is to be stated that the described ophthalmologic apparatus 1 enables a breaking down of eye tissue as a three-dimensional ablation process, the effects illustrated in FIGS. 3a, 3b, 4a, 4b, 4c, 5a, 5b and 6 being respectively illustrated in plan view.

1. An ophthalmologic apparatus comprising:
a light projector for projecting femtosecond laser pulses for breaking down eye tissue, respectively focused onto a focal surface, wherein the apparatus further comprises optical means for producing pulse processing areas that are respectively formed on the focal surface by one of the femtosecond laser pulses and respectively have an area boundary on the focal surface that deviates from a circular shape.

2. The apparatus as claimed in claim 1, further comprising positioning means for positioning the pulse processing areas along a processing line, the optical means being configured to align the pulse processing areas with reference to the processing line.

3. The apparatus as claimed in claim 2, wherein the optical means are configured to align the pulse processing areas with reference to the processing line such that a longitudinal axis running in a relatively wide extent of the pulse processing area is respectively perpendicular to the processing line.

4. The apparatus as claimed in claim 2, wherein the optical means are configured to align the pulse processing areas with reference to the processing line such that a transverse axis running in a relatively narrow extent of the pulse processing area is respectively perpendicular to the processing line.

5. The apparatus as claimed in claim 2, further comprising a control module for controlling the optical means in such a way that the optical means align the pulse processing areas with reference to the processing line in accordance with a control value.

6. The apparatus as claimed in claim 2, further comprising a control module for controlling the optical means in such a way that the optical means produce the shape of the area boundary of the pulse processing areas in accordance with a control value.

7. The apparatus as claimed in claim 2, wherein the positioning means and the optical means are configured to position and/or to produce the pulse processing areas such that successive pulse processing areas at least partially overlap.

8. The apparatus as claimed in claim 1, wherein the optical means are configured to produce the pulse processing areas with a substantially elliptically shaped area boundary.

9. The apparatus as claimed in claim 1, wherein the area boundary is defined by a limit value for a drop in intensity of one of the femtosecond laser pulses on the focal surface.

10. The apparatus as claimed in claim 1, wherein the optical means for producing the pulse processing areas comprise at least one of the following: polarization modulator, phase modulator and intensity modulator.

11. The apparatus as claimed in claim 1, wherein the optical means for producing the pulse processing areas comprise at least one of the following: anamorphotic optical module, deformable mirror, photonic crystal, photonic bandgap fiber, diffractive optical module, lens array, polarization filter, spatial polarization plate, λ-half-plate, stop and spatial light modulator.

12. The apparatus as claimed in claim 1, wherein the apparatus is configured to produce incisions in the eye tissue.
